(19) United States
(12) Patent Application Publication
(10) Pub. No.: US 2026/0053402 A1
(43) Pub. Date: Feb. 26, 2026

(54) BIOSENSOR AND METHODS OF BIOSENSOR CONSTRUCTION

(71) Applicant: Konamite Limited, Dublin (IE)

(72) Inventor: Mihailo V. Rebec, Portland, OR (US)

(21) Appl. No.: 19/305,247

(22) Filed: Aug. 20, 2025

Related U.S. Application Data
(60) Provisional application No. 63/685,300, filed on Aug. 21, 2024.

(51) Int. Cl. A61B 5/1486; A61B 5/145; B23K 26/36

(52) U.S. Cl. CPC A61B 5/14865; A61B 5/14532; B23K 26/36; A61B 2562/12

(57) ABSTRACT

Embodiments provide for an analyte sensors and methods of construction thereof. In one example, a method of constructing an indwelling analyte sensor comprises coating a wire with a dielectric material, removing one or more regions of the dielectric material to expose one or more electrochemically active surface(s), producing a pattern in the one or more exposed electrochemically active surfaces to increase surface area of the one or more electrochemically active surfaces, and coating remaining regions of dielectric material and the one or more exposed electrochemically active surfaces with a membrane material comprising an enzyme layer. In this way, sensor response and performance can be improved with corresponding reductions in in-skin sensor length.

### CROSS-REFERENCE TO RELATED APPLICATIONS

This application claims the benefit of U.S. Provisional Application No. 63/685,300, filed on Aug. 21, 2024. The disclosure of the prior application is hereby incorporated by reference in its entirety.

### TECHNICAL FIELD

Embodiments herein relate to the field of analytical devices used for detection of a chemical substance, and, more specifically, to continuous glucose monitoring devices.

### BACKGROUND

Continuous monitoring devices, such as continuous glucose monitoring (CGM) devices, are used to measure data continuously, for example in a continuous data stream and/or sampled data points over a time interval. Self-monitoring of glucose via the use of CGM devices for individuals afflicted with diabetes mellitus enables patients to take control of their disease, and thus directly affect outcomes related to it. CGM devices rely on a sensor that is inserted into skin of a patient, and for accurate and reliable results, performance of the sensor is critical. Issues related to non-optimal sensor performance contribute to the reduced adoption and durability of CGM device usage. Accordingly, for increased uptake and sustained use of the technology, there is a need for improved sensor performance characteristics.

### SUMMARY

An aspect of this disclosure includes a method of constructing an indwelling analyte sensor, comprising applying a dielectric material to a wire comprising electrochemically active surfaces; removing one or more selected regions of the applied dielectric material to expose one or more electrochemically active surfaces of the electrochemically active surfaces of the wire; producing a pattern in at least one of the exposed one or more electrochemically active surfaces; and applying a membrane material to remaining regions of the dielectric material and the exposed one or more electrochemically active surfaces, the membrane material comprising an enzyme layer.

In some embodiments, the indwelling analyte sensor comprises a glucose sensor; and the enzyme layer comprises a glucose oxidase layer. In some embodiments, the dielectric material comprises at least one of a polyimide material, a biocompatible solder mask material, an epoxy acrylate copolymer material, a polyurethane material, or a parylene material. In some embodiments, removing the one or more selected regions of the dielectric material comprises at least one of manual removal, laser-ablation, chemical etching, or grit-blasting the one or more selected regions of the dielectric material to expose the one or more electrochemically active surfaces of the wire. In some embodiments, the wire comprises a tantalum core wire coated with a platinum iridium layer. In some embodiments, producing the pattern comprises performing a laser-ablation operation of the platinum iridium layer corresponding to at least one of the one or more electrochemically active surfaces. In some embodiments, performing the laser-ablation operation comprises laser-ablating the platinum iridium layer with a femtosecond laser source.

In some embodiments, performing the laser-ablation operation comprises rotating and indexing the wire. In some embodiments, producing the pattern comprises creating a plurality of grooves in the electrochemically active surface. In some embodiments, each of the plurality of grooves has a depth of at least 20 nm. In some embodiments, applying the membrane material comprises brush coating the remaining regions of the dielectric material and the one or more electrochemically active surfaces with the enzyme layer. In some embodiments, the pattern increases a sensitivity per mm of the one or more exposed electrochemically active surfaces as compared to electrochemically active surfaces lacking the pattern.

Another aspect of this disclosure includes an indwelling analyte sensor, comprising an electrochemically active surface coated with a dielectric material; at least one cavity region in which the electrochemically active surface is free of the dielectric material and wherein the electrochemically active surface is of a patterned texture in one or more of the at least one cavity regions, the patterned texture increasing a surface area of the electrochemically active surface in the at least one cavity region as compared to a non-patterned electrochemically active surface in the at least one cavity region; and a membrane material comprising an enzyme layer, the enzyme layer surrounding the dielectric material and the electrochemically active surface corresponding to the at least one cavity region.

In some embodiments, the dielectric material is a polyimide material. In some embodiments, the electrochemically active surface comprises a platinum iridium layer. In some embodiments, the platinum iridium layer surrounds a tantalum core wire. In some embodiments, the membrane material includes multiple membranes. In some embodiments, an in-skin length of the sensor is 7 mm or less. In some embodiments, the patterned texture comprises one or more of a ribbed texture, a ridged texture, and a dimpled texture.

Another aspect of this disclosure includes a glucose biosensor, comprising a set of two terminal nubs of dielectric material extending outward from and spaced along an electrochemically active surface; one or more cavity regions defined by an absence of dielectric material, the one or more cavity regions positioned between the set of two terminal nubs; a membrane material comprising a glucose oxidase layer that surrounds the set of two terminal nubs and the electrochemically active surface; and wherein the electrochemically active surface extends through at least a portion of the set of two terminal nubs, and has a patterned surface texture in at least the one or more cavity regions, the patterned surface texture increasing a surface area of the electrochemically active surface corresponding to the one or more cavity regions as compared to an electrochemically active surface lacking the patterned surface texture.

In some embodiments, the electrochemically active surface comprises a platinum iridium layer surrounding a tantalum core wire; and wherein the platinum iridium layer comprises the patterned surface texture. In some embodiments, the one or more cavity regions each have a length of 0.025 mm-3 mm. In some embodiments, the membrane material has an outer surface, the outer surface defining a concave curve curving toward the electrochemically active surface in the one or more cavity regions; and wherein the membrane material includes multiple membranes and defines an external surface of the biosensor. In some embodiments, a plurality of annular plates spaced between the set of two terminal nubs. In some embodiments, the one or more cavity regions are positioned (i) between at least two of the plurality of annular plates and (ii) between at least two of the plurality of annular plates and the set of two terminal nubs; the glucose oxidase layer surrounds the plurality of annular plates; and the electrochemically active surface extends through at least two of the plurality of annular plates.

Another aspect of this disclosure includes an indwelling analyte sensor, comprising: an electrochemically active surface devoid of a dielectric material; at least one cavity region in which the electrochemically active surface is of a patterned texture, wherein the patterned texture increases a surface area of the electrochemically active surface in the at least one cavity region relative to a corresponding non-patterned electrochemically active surface; and a membrane material comprising an enzyme layer that surrounds the electrochemically active surface in the at least one cavity region, wherein the patterned texture of the electrochemically active surface increases a uniformity of the enzyme layer surrounding the electrochemically active surface relative to a non-patterned texture. In some embodiments, the uniformity of the enzyme layer corresponds to a smoothness of the enzyme layer surrounding the electrochemically active surface.

### BRIEF DESCRIPTION OF DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1A depicts a side view of a work piece formed as part of the construction of a biosensor using methods of the present disclosure.

FIG. 1B depicts a side view of a sensor that can be constructed from the work piece of FIG. 1A.

FIG. 1C depicts a side-view of a portion of a non-patterned work piece of the present disclosure.

FIG. 1D depicts a side-view of a portion of a patterned work piece of the present disclosure.

FIG. 1E depicts another view of a non-patterned work piece of the present disclosure.

FIG. 1F depicts another view of a patterned work piece of the present disclosure.

FIG. 1G depicts yet another side-view of a patterned work piece of the present disclosure.

FIGS. 2A-2D depict partial side views of example work pieces formed as part of the construction of a biosensor using methods of the present disclosure.

FIG. 3A depicts another side view of a work piece formed as part of the construction of a biosensor using methods of the present disclosure.

FIG. 3B depicts a side view of a sensor that can be constructed from the work piece of FIG. 3A.

FIG. 4A depicts an example illustration of a sensor with a first insertion length, inserted at a first insertion angle.

FIG. 4B depicts an example illustration of a sensor with a second insertion length, inserted at a second insertion angle.

FIG. 5 depicts a high level example system for constructing work pieces of the present disclosure.

FIG. 6 depicts a high level example method for construction of a sensor, according to an embodiment of the present disclosure.

FIG. 7 depicts another side view of a work piece formed as part of the construction of a biosensor using methods of the present disclosure.

FIG. 8 depicts an image of a work piece formed as part of the construction of a biosensor using methods of the present disclosure.

FIG. 9A depicts an image of a portion of a work piece formed as part of the construction of a biosensor using methods of the present disclosure.

FIG. 9B depicts a dimensional measurement pattern of part of the work piece of FIG. 9A.

### DETAILED DESCRIPTION

In the following detailed description, reference is made to the accompanying drawings which form a part hereof, and in which are shown by way of illustration embodiments that may be practiced. It is to be understood that other embodiments can be utilized, and structural or logical changes can be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide for analyte sensors and methods of their construction. The analyte sensors disclosed herein can be used by a subject (e.g., a human or mammal) to detect analytes (e.g., glucose) in interstitial fluid. In one example, a method of constructing an indwelling analyte sensor comprises coating a wire with a dielectric material, removing one or more selected regions of the dielectric material to expose one or more electrochemically active surface(s) of the wire thereby providing a work piece, producing a pattern in at least one of the one or more electrochemically active surfaces that have been exposed, and coating remaining regions of dielectric material and the one or more electrochemically active surfaces that have been exposed with a membrane material comprising an enzyme layer. In such an embodiment, the pattern may increase a surface area of the electrochemically active surface as compared to electrochemically active surface lacking the pattern, which in turn may improve sensor performance. For example, sensor response may be improved by 2-3 times or more (e.g., 2 times, or 3 times, or 4 times, or 5 times, or 6 times) over a similarly constructed sensor that does not include the pattern in the electrochemically active surface. The pattern may be created via a laser-ablation operation, such as a laser that utilizes a laser pulse (e.g., femtosecond laser pulse) to produce the pattern (e.g., ribbed/ridged/dimpled pattern as elaborated below).

In another embodiment, an indwelling analyte sensor comprises an electrochemically active surface coated with a dielectric material. The sensor may further comprise at least one cavity region in which the electrochemically active surface is free of the dielectric material and wherein the electrochemically active surface is of a patterned texture in one or more of the at least one cavity regions. The patterned texture may increase a surface area of the electrochemically active surface in one or more of the at least one cavity regions, as compared to an electrochemically active surface of otherwise same dimensions lacking the patterned texture. The sensor may further comprise a membrane material comprising an enzyme layer, the enzyme layer surrounding the dielectric material and the electrochemically active surface corresponding to the at least one cavity region.

In yet another embodiment, a glucose biosensor comprises a plurality of annular plates of dielectric material and a set of two terminal nubs extending outward from and spaced along an electrochemically active surface. The biosensor may further comprise one or more cavity regions (e.g., 1, 2, 3, 4, 5, 6, 7, or 8 cavity regions) defined by an absence of dielectric material, the one or more cavity regions positioned between at least two of the plurality of annular plates and between at least two of the plurality of annular plates and the set of two terminal nubs. The biosensor may further comprise a membrane material comprising a glucose oxidase layer that surrounds the plurality of annular plates, the set of two terminal nubs, and the electrochemically active surface. In such an embodiment, the electrochemically active surface may extend through at least two of the plurality of annular plates and through at least a portion of the set of two terminal nubs, and may have a patterned surface texture in at least the one or more cavity regions.

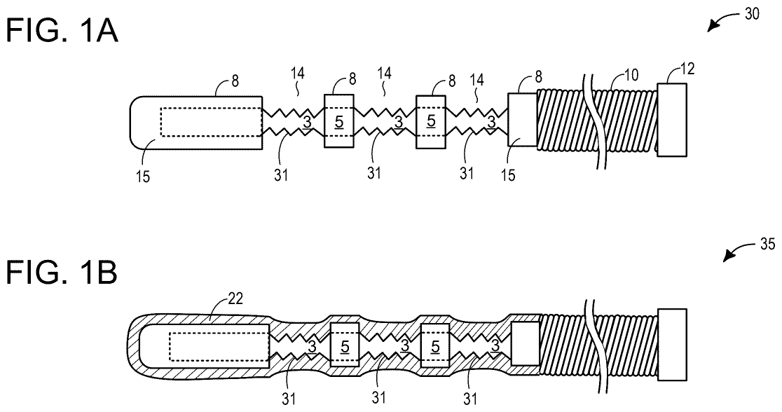

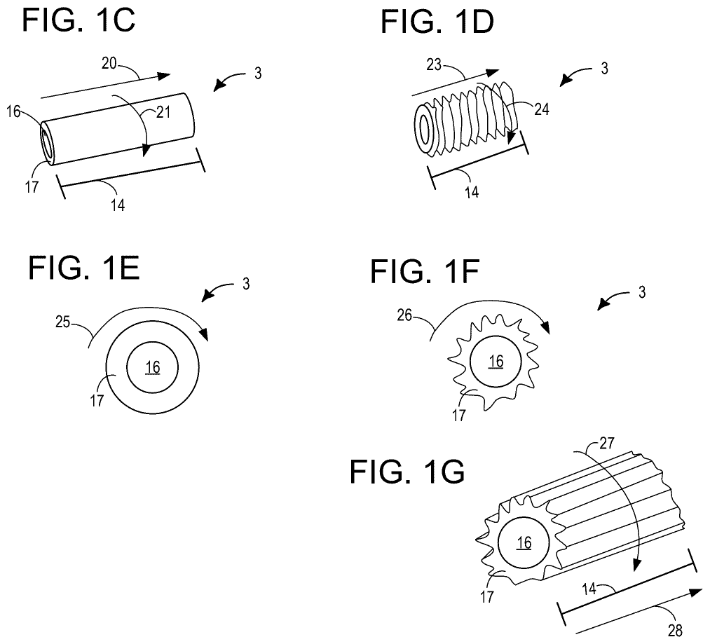

Turning to the Figures, FIG. 1A depicts work piece 30, including a wire 3. Wire 3 may be a comprised of a platinum iridium layer coated on a tantalum core, for example. However, such an example is not meant to be limiting. For example, stainless steel, titanium, a conductive polymer (polypyrrole [PPy], polyaniline, PEDOT, polyacetylene, polyphenylene vinylene, polythiophene, polyphenylene sulfide, etc.), an alloy, or the like may be used as alternatives to tantalum. Furthermore, the core may be coated and/or plated with conductive material comprising platinum, platinum-iridium, gold, palladium, iridium, graphite, carbon, a conductive polymer, or combinations thereof. The coating is referred to herein as an electrochemically active surface layer. The electrochemically active surface layer may be between 1-30 μm thick. In some embodiments, the electrochemically active surface layer may be greater than 30 μm thick, such as between 30-35 μm thick, or 35-40 μm thick, or 40-45 μm thick, or 45-50 μm thick. In some embodiments, a diameter of wire 3, including core and electrochemically active surface layer may be between 20 μm or less to 250 μm or greater.

Wire 3 may be coated with an insulating layer 8. Insulating layer 8 (also referred to herein as dielectric layer 8, or dielectric material 8) may comprise a polyimide layer, for example. Other examples of insulating layer 8 can include, but are not limited to biocompatible solder masks, epoxy acrylate copolymers, polyurethane, parylene, or the like. The insulating layer 8 may be 5-30 μm in thickness (for example, between 15 μm and 30 μm). A reference electrode, such as a silver (Ag/AgCl) wire, 10, may be wrapped about at least a portion of insulating layer 8. In other embodiments, the reference electrode 10 may be comprised of a silver-containing polymer, silver-containing ink, silver-containing paint, silver-containing paste, or the like. In some embodiments, Ag/AgCl particles may be mixed into a polymer, such as polyurethane, polyimide, or the like, to form the silver-containing material for reference electrode 10. Work piece 30 may be used to form an analyte sensor such as analyte sensor 35 depicted at FIG. 1B, which will be discussed in greater detail below. A housing 12 (which can be overmolded from plastic or any other suitable material) may house one or more contacts that couple with the sensor anode, the sensor cathode, and/or the inner wire 3.

Cavities 14 (also referred to herein as cavity regions 14) may be formed by laser ablating insulating layer 8, thereby forming work piece 30 as depicted at FIG. 1A. In other embodiments, cavities 14 may be formed by chemical etching, grit-blasting, excimer lasing, manual removal, etc. Each of cavities 14 may be 0.025-3 mm long. In some embodiments, one or more of cavities 14 may be 0.025 mm-0.05 mm in length, or 0.05-0.1 mm in length, or 0.1 mm in length to 0.2 mm in length, or 0.25-0.5 mm in length, or 0.5-1 mm in length, or 1-1.5 mm in length, or 1.5-2 mm in length, or 2-2.5 mm in length, or 2.5-3 mm in length, or 2.5-3 mm in length. In particular examples, each of cavities 14 are 0.025 mm in length, or 0.05 mm in length, or 0.1 mm in length, or 0.125 mm in length, or 0.15 mm in length, or 0.2 mm in length, 0.25 mm in length, or 0.3 mm in length, or 0.35 mm in length, or 0.4 mm in length, or 0.45 mm in length, or 0.5 mm in length, or 0.55 mm in length, or 0.6 mm in length, or 0.65 mm in length, or 0.7 mm in length, or 0.75 mm in length, or 0.8 mm in length, or 0.85 mm in length, or 0.9 mm in length, or 0.95 mm in length, or 1 mm in length, or 1.1 mm in length, or 1.2 mm in length, or 1.3 mm in length, or 1.4 mm in length, or 1.5 mm in length, or 1.6 mm in length, or 1.7 mm in length, or 1.8 mm in length, or 1.9 mm in length, or 2.0 mm in length, or 2.1 mm in length or 2.2 mm in length or 2.3 mm in length, or 2.4 mm in length, or 2.5 mm in length, or 2.6 mm in length, or 2.7 mm in length, or 2.8 mm in length, or 2.9 mm in length, or 3.0 mm in length, or 3.5 mm in length. In some embodiments, one or more of cavities 14 are about 0.125 mm in length, about 0.3 mm in length, about 0.75 mm in length, or about 2.0 mm in length. In other embodiments, one or more of cavities 14 are about 0.5 mm in length, about 0.83 mm in length, about 1.5 mm in length, or about 3.5 mm in length.

The insulating layer 8 that remains between cavities 14 forms a set of annular plates 5. Annular plates 5, as discussed herein, refer to regions of insulating layer 8 bounded on two sides by cavities 14. Dielectric material 8 which remains following removal (e.g. laser ablation) but which includes a cavity 14 on just one side is herein referred to as a nub 15, nubs 15, or terminal nubs 15. Each of annular plates 5 and nubs 15 are supported by adherence of the insulating layer 8 onto wire 3. In the example depicted at FIG. 1A, removal of the insulating layer 8 (e.g., via laser ablation) does not ablate any portion of wire 3 in the formation of work piece 30 but see below for other embodiments. Annular plates 5 can be spaced at regular intervals or irregular intervals along the wire 3 (e.g., between the nubs 15). Annular plates 5 (also referred to herein as plates 5) can be formed by removing portions of insulating layer 8 to create the one or more of cavities 14 as described herein and depicted in FIG. 1A.

Each of the annular plates 5 can be of various widths. In some embodiments, one or more of the annular plates 5 can be about 0.05 mm to about 5.0 mm. For example, the annular plate 5 can comprise a width of about 0.05 mm to about 4.5 mm, about 0.1 mm to about 4.0 mm, about 0.15 mm to about 3.5 mm, about 0.2 mm to about 3.0 mm, about 0.25 mm to about 3.0 mm, about 0.3 mm to about 3.0 mm, about 0.3 mm to about 3.0 mm, about 0.35 mm to about 3.0 mm, about 0.4 mm to about 3.0 mm, about 0.45 mm to about 3.0 mm, about 0.5 mm to about 3.0 mm, about 0.55 mm to about 3.0 mm, about 0.6 mm to about 3.0 mm, about 0.65 mm to about 3.0 mm, about 0.7 mm to about 3.0 mm, about 0.75 mm to about 3.0 mm, about 0.8 mm to about 3.0 mm, about 0.85 mm to about 3.0 mm, about 0.9 mm to about 3.0 mm, about 0.95 mm to about 3.0 mm, about 1.0 mm to about 3.0 mm, about 1.25 mm to about 3.0 mm, about 1.5 mm to about 3.0 mm, about 1.75 mm to about 3.0 mm, about 2.0 mm to about 3.0 mm, about 2.5 mm to about 3.0 mm, or about 2.75 mm to about 3.0 mm. In some embodiments, one or more of the annular plates comprise a width of about 0.5 mm.

In a subsequent step following removal of the insulating layer 8, a laser ablation operation may be used to create a patterning (e.g., surface modification) 31 in the electrochemically active surface of wire 3 (depicted at FIG. 1A as a sawtooth shape). The surface modification may increase electrochemically active surface area as compared to a non-patterned electrochemically active surface layer, which may thereby improve sensor performance for reasons elaborated below.

As discussed herein, “patterning” of an electrochemically active surface is defined as a surface modification of an electrochemically active surface such that a cross section of wire 3 has altered dimensions (e.g., varying) when measured along a particular unit length (e.g., length of cavity region 14) and/or as measured along a circumference of wire 3. This is in contrast to a non-patterned electrochemically active surface that has a circular cross-section of substantially same dimensions (e.g., non-varying) along an entirety of a particular unit length (e.g., cavity region 14) and circumference of wire 3. The altered cross-section along the unit length (e.g., cavity region 14) and/or circumference of a patterned electrochemically active surface as defined herein may comprise a ridged, ribbed, irregular, dimpled, or otherwise altered cross-sectional dimensions as compared to a uniformly circular cross-section as measured along a length and circumference of a non-patterned wire 3. For reference, FIG. 1C depicts a side-view of cavity region 14 comprising wire 3 with core 16 (e.g., tantalum core) and electrochemically active surface 17 (e.g., platinum iridium surface), where the electrochemically active surface is non-patterned. Specifically, a cross-section of wire 3 as depicted at FIG. 1C that comprises a non-patterned electrochemically active surface is unaltered when measured along a length (refer to arrow 20 at FIG. 1C) and circumference (refer to arrow 21 at FIG. 1C) corresponding to cavity region 14. In contrast, FIG. 1D depicts a side view of a portion of wire 3 comprising core 16 and wherein electrochemically active surface 17 has been patterned via the methodology disclosed herein. As illustrated, the cross-section is altered (e.g., varies) along a length (refer to arrow 23 at FIG. 1D) corresponding to cavity region 14. Cross-sectional dimensions may also vary at least somewhat as measured along a circumference (refer to arrow 24 at FIG. 1D) of wire 3 as depicted for FIG. 1D.

FIG. 1E depicts another view (e.g., frontal view as opposed to side-view) of wire 3 for which electrochemically active surface 17 is non-patterned along a circumference (refer to arrow 25) of wire 3. Alternatively, FIG. 1F depicts a frontal view similar to that of FIG. 1E of wire 3 for which electrochemically active surface 17 has been patterned via methodology of the present disclosure. As depicted, the cross-section of wire 3 at FIG. 1F is clearly altered along at least a circumference (refer to arrow 26) of wire 3. FIG. 1G depicts a side-view of the same wire 3 depicted at FIG. 1F, to illustrate how the patterning is altered along the circumference (refer to arrow 27) of wire 3. In such an example as that depicted at FIGS. 1F-1G, cross-sectional dimensions can also vary along a length (refer to arrow 28) of wire 3. As discussed herein, an altered cross-section, or varying cross-section may comprise a non-constant cross-sectional dimensions when measured along a length and/or circumference of a wire 3 that has a patterned electrochemically active surface.

According to some embodiments, the patterning 31 may be created via a femtosecond laser source combined with a rotating and indexing stage so as to create a desired patterning in the platinum surface of wire 3. Examples of femtosecond laser sources relevant to the present disclosure include but are not limited to solid-state bulk lasers (e.g., diode-pumped lasers, titanium-sapphire lasers, etc.), fiber lasers (e.g., ultrafast fiber lasers, stretched-pulse fiber lasers, similariton lasers, fiber lasers in combination with a fiber amplifier, etc.), dye lasers, semiconductor lasers (e.g., mode-locked diode lasers, passively mode-locked vertical external-cavity surface-emitting lasers (VECSELs), etc.), free electron lasers, etc. Laser-machining operations to create patterning 31 may comprise lasing a particular electrochemically active surface along a length of, for example, a cavity region 14 and/or or along a circumference corresponding to, for example, a cavity region 14. For example, a laser-machining operation may include lasing along a length of a cavity region, then circumferentially rotating wire 3 by a defined amount (or repositioning the laser source) and again lasing another portion of the electrochemically active surface along the same length. This type of laser-machining (e.g., laser etching) operation may be repeated any number of times until the electrochemically active surface is patterned as desired. Such a laser-machining operation may produce a patterning similar to that depicted at FIG. 1G.

In another example, a laser machining operation may comprise lasing along a circumference of an electrochemically active surface, and simultaneously indexing wire 3 so as to achieve a patterning that approximates a spiraling pattern around a circumference of the electrochemically active surface layer, along a length of, for example, a cavity region 14. As another example, a laser-machining operation may include lasing around an entirety of a first circumference of an electrochemically active surface, then indexing the wire 3 a determined amount, and repeating the laser-machining operation along an entirety of a second circumference of the electrochemically active surface. Such an operation may be repeated any number of times until the electrochemically active surface is patterned as desired. Such laser-machining operations may produce patterning similar to that depicted at FIG. 1D.

In some embodiments, the ablation of the insulating layer 8 and/or the electrochemically active surface to create patterning 31 can be carried out using a laser skiving system configured for rotation and translation of the wire 3 during ablation. One example of such a system is a UV laser skiving platform, such as those commercially available from HYLAX TECHNOLOGY, although other laser systems capable of producing similar cavity geometries and patterning are also contemplated.

In yet another example, it is within the scope of this disclosure to employ a laser machining operation first lasing along a circumference of an electrochemically active surface as discussed above, and then lasing along a length of the electrochemically active surface, to create a wavy patterning in the electrochemically active surface. Such a wavy patterning may result in altered cross-sectional dimensions as measure along both a length and a circumference of, for example, a cavity region 14 of wire 3.

Returning to FIG. 1B, following the laser-machining operation to create the desired patterning, the work piece may be coated with a membrane material 22 that permits detection of an analyte (e.g., glucose) via sensor 35. In an example, membrane material 22 may be comprised of a set of layers that are constructed through a coating and curing process, including a sequence of dip coating operations interspersed with curing operations. These layers may include an interferent excluding layer, a glucose oxidase layer, and a permselective layer as described in U.S. Pat. No. 5,165,407, which is hereby incorporated by reference in its entirety. In some embodiments, the set of layers includes two layers: an outer layer (e.g., a second layer) that includes interferent excluding material and permselective material; and a first layer that includes enzyme material. The set of layers may undergo a plasma treatment operation that modifies the platinum and/or insulating layer(s). The work piece may be coated with membrane material 22 via dip-coating, spray coating, spin-coating, selective deposition methodology, or the like. In some embodiments, the work piece is dip-coated.

According to some embodiments, due to the inclusion of the annular plates 5 and nubs 15, the dip coating may result in a surface of the viscous fluid comprising the material 22 forming a shape similar to a catenary curve between annular plates 5 and/or between nubs 15 and annular plates 5. As such, a greater portion of viscous fluid may adhere than would otherwise adhere in absence of annular plates 5. Inclusion of a greater number of annular plates (e.g., 3, 4, 5, 6) for forming sensor 35 may correspondingly reduce a length of each of cavity regions 14. The closer annular plates 5 are to one another (or the closer a nub 15 to an annular plate 5), the shallower the curvature of the surface of the viscous fluid comprising the material 22 between the annular plates 5 may be. In some embodiments where annular plates 5 are sufficiently close to one another, the curvature may be eliminated. For example, the curvature may be eliminated or substantially eliminated under conditions where annular plates 5 (or nubs 15 and annular plates 5) are 0.025-0.05 mm apart, or 0.05-0.1 mm apart, or 0.25-0.3 mm apart, or 0.3-0.35 mm apart, or 0.35-0.4 mm apart, or 0.4-0.45 mm apart, or 0.45-0.5 mm apart, or 0.5-0.55 mm apart, or 0.55-0.6 mm apart, or 0.6-0.65 mm apart, or 0.65-0.7 mm apart, or 0.7-0.75 mm apart, or 0.75-0.8 mm apart, or 0.8-0.85 mm apart, or 0.85-0.9 mm apart, or 0.9-0.95 mm apart, or 0.95-1 mm apart. Furthermore, as annular plates 5 (or nubs 15 and annular plates 5) become closer to one another, a variability in terms of amount of viscous fluid that adheres to cavity regions 14 may be correspondingly reduced. It is herein recognized that such a reduction in variability of amount of viscous fluid that adheres to cavity regions 14 may translate into advantages in terms of improved sensitivity and performance of the sensors of the present disclosure, and may reduce overall variability in terms of sensor response properties between, for example, one sensor and another.

In some embodiments, sensor sensitivity to chemical substances (e.g., glucose) can be improved by varying the number and lengths of the cavities 14. For example, the number and length and length of the cavities 14 can be varied by increasing or decreasing the number of annular plates 5 and/or their distance relative to each other within the analyte sensing region. In some embodiments, a workpiece 30 can be manufactured with more than one cavity 14. In such embodiments, the spacing between annular plates 5 (e.g., the spacing between adjacent plates of the annular plates 5) and/or between a nub 15 and an adjacent annular plate 5 can influence the geometry and distribution of the membrane material 22 that surrounds the annular plates 5, nubs 15, and the electrochemically active surface of wire 3. For example, when a workpiece 30 is configured to have cavities 14 with shorter lengths (e.g., lengths of about 0.125 mm to about 1.5 mm) the annular plates 5 and/or nubs 15 are positioned closer depending on the quantity of cavities and length of the working electrode region (cavity regions 14 and annular plates 5).

In some embodiments, during the coating and curing process, when the annular plates 5 are closer to each other (or closer to a nub 15), the distribution of membrane material 22 results in a thinner coating formed between adjacent annular plates 5 (or between a nub 15 and an adjacent annular plate 5). For example, when the wire 3 is dip-coated with the layers of the membrane material 22, closely spaced nubs 5 affect how the membrane material 22 dries. Said differently, the membrane material 22 becomes thinner between nearby nubs 5, and this thinner membrane material 22 can allow chemical substances (e.g., glucose) to pass through with improved frequency. Accordingly, embodiments that reduce individual cavity length 14 while holding total sensing region length approximately constant can yield a higher sensitivity/mm due to changes in membrane material 22 deposition.

For example, consistent with certain embodiments, an analyte sensor 35 can include a 3.5 mm working electrode region with four cavities 14 that are 0.5 mm in length, separated by three annular plates 5 that are 0.5 mm in width. In this example, the close spacing between adjacent annular plates 5 influences the geometry of membrane material 22 during the coating and curing process (e.g., coating and drying steps of the coating and curing process), resulting in a thinner membrane layer formed between the annular plate structure 5 (or the annular plate structure 5 and the nub 15). This membrane material 22 thinning can be the result of surface tension and capillary effects acting on the viscous membrane material 22 as it spans the narrow gaps between the structures of the annular plates 5. The thinner membrane material 22 formed in this manner can allow increased analyte flux to the electrochemically active surface of wire 3, thereby enhancing signal generation within each cavity region 14. As a result, the sensor 35 can exhibit an increased sensitivity per millimeter of cavity 14 length, even when each individual cavity 14 is relatively short and/or the surface area is decreased. This configuration allows for enhanced signal response while maintaining a compact sensor length. For instance, an analyst sensor 35 having a 3.5 mm working electrode region and four cavities 14 that are 0.5 mm in length can exhibit a six-fold increase in sensitivity/mm as compared to an analyte sensor 35 with a 3.5 mm working electrode region with one cavity 14 that is 3.5 mm in length.

In another example, consistent with certain embodiments, an analyte sensor 35 can include a 2.0 mm working electrode region with four cavities 14 that are 0.125 mm in length, separated by three annular plates 5 that are 0.5 mm in width. In this example, the close spacing between adjacent annular plates 5 also influences the geometry of membrane material 22 during the coating and curing process (e.g., coating and drying steps of the coating and curing process), resulting in a thinner membrane layer formed between the annular plate structure 5 (or the annular plate structure 5 and the nub 15). As explained above, this membrane material 22 thinning can be the result of surface tension and capillary effects acting on the uncured membrane material 22 as it spans the narrow gaps between the structures of the annular plates 5. The thinner membrane material 22 formed in this manner can allow increased analyte flux to the electrochemically active surface of wire 3, thereby enhancing signal generation within each cavity region 14. As a result, the sensor 35 can exhibit an increased sensitivity per millimeter of cavity 14 length, even when each individual cavity 14 is relatively short and/or the surface area is decreased. For instance, an analyst sensor 35 having a 2.0 mm working electrode region and four cavities 14 that are 0.125 mm in length can exhibit a thirteen-fold increase in sensitivity/mm as compared to an analyte sensor 35 with a 2.0 mm working electrode region with one cavity 14 that is 2.0 mm in length.

Achieving high sensitivity while reducing the length of the subcutaneous portion of the analyte sensor can provide advantages for insertion and placement of the analyte sensor on a skin surface of a subject. In some embodiments, analyte sensors 35 of the present disclosure can be configured for perpendicular (e.g., approximately 90°) insertion into the skin surface. Such an insertion orientation may be desirable for particular anatomical locations, user preference, specific applicator designs, or applications involving shallow analyte targets. To accommodate a perpendicular insertion while avoiding deeper tissue contact, the subcutaneous length of the sensor 35 can be reduced. In these configurations, sensitivity can be maintained or enhanced by including multiple cavity regions 14 of reduced length, spaced apart by annular plates 5 and/or nubs 15. For example, a sensor with an indwelling length of approximately 2 mm can include four cavity regions 14, each approximately 0.125 mm in length, separated by three polyimide nubs 15 of 0.5 mm width. In another example, a sensor configured with a 3.5 mm sensing region for perpendicular insertion can include three cavity regions 14 of approximately 0.833 mm in length, separated by two nubs 15 of 0.5 mm width. In such examples, the close spacing of the polyimide features can result in membrane material 22 thinning between adjacent annular plates 5 and/or nubs 15, which may increase analyte flux and signal magnitude from each cavity region 14. Accordingly, perpendicular insertion configurations can be supported without requiring increased exposed surface area or overall indwelling length, enabling compact sensors with high signal output.

As mentioned, the surface patterning (e.g., patterning 31 at FIGS. 1A-1B) may increase reaction surface area as compared to a non-patterned surface of wire 3. Specifically, discussed herein cavity regions 14 correspond to areas of exposed electrochemical area where response to the presence of analyte occurs. As an example, such a region in a glucose sensor may be an area where hydrogen peroxide is converted to water and electrons.

Such an increase in reaction surface area may improve sensor performance and may be advantageous as compared to other options for increasing sensor performance. For example, other less-advantageous options for increasing reaction surface area may include increasing cavity areas in one of two ways. In another example, an increase in cavity area may be achieved by increasing a length of the sensor so as to accommodate a greater number of cavity regions 14 of a particular length, and hence a greater electrochemically active surface area. However, while an increase in sensor length to accommodate increased exposed electrochemical surface area may improve sensor performance by increasing reaction surface area, disadvantages may include a reduction in options for areas on the body where the sensor may be applied.

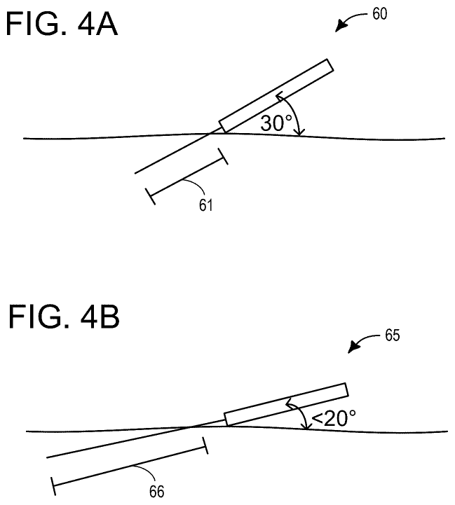

Turning briefly to FIGS. 4A-4B, FIG. 4A depicts a first sensor 60 with a first indwelling length 61, and FIG. 4B depicts a second sensor 65 with a second indwelling length 66. As depicted, the first indwelling length 61 is of a shorter length than the second indwelling length 66. Accordingly, the first sensor 60 depicted at FIG. 4A may be inserted into skin at an insertion angle of 300 or greater, whereas the second sensor depicted at FIG. 4B may have to be inserted into skin at an insertion angle of 20° or less, for a same body location to avoid detrimental aspects (e.g., insertion trauma) associated with insertion. Accordingly, while a longer sensor length may enable improved performance, the performance improvement may come with a reduced number of locations where the sensor can be effectively accommodated. The sensor length may vary based on the analyte being tracked and where the analyte is being sampled. For example, if the analyte is found in the interstitial fluid, a shorter sensor and shallower depth (e.g., 1-7 mm) can be used. If lactic acid buildup is being tracked (e.g., during exercise), the sensor would have to be long enough (e.g., up to 15 mm or longer) to reach the muscle tissue. Further, for sensors that do not rely on a trocar, such as the sensors of some embodiments of the present disclosure, reduced angle of insertion due to increased sensor length may increase a potential that the sensor may become dislodged (e.g., skip out of the skin surface) upon insertion or during use. By creating the patterning (e.g., surface modification) 31 in the electrochemically active surface (e.g., platinum iridium surface) of wire 3, reaction surface area may be increased without a corresponding increase in in-skin sensor length, nor a thinning of membrane material 22.

Returning now to FIG. 1A, illustrated is work piece 30 where each region of exposed electrochemically active surface associated with a cavities 14 have been laser ablated to create patterning 31. In other examples, select cavities 14 may be laser ablated to create patterning 31, whereas other cavities 14 may not be laser ablated (e.g., absence of patterning in platinum surface), without departing from the scope of this disclosure. Patterning of the electrochemically active surface may increase the active area of a cavity region 14 by 1.5-4-fold or greater (e.g., 2-fold, 3-fold, 4-fold, 5-fold, or 6-fold) over the active area of a similarly sized cavity region 14 that lacks patterning 31 (e.g., cavity region of same length and outermost diameter).

Based on the above-mentioned ranges in terms of length of a cavity region 14 corresponding to an electrochemically active region, and diameter of wire 3, surface area of an unpatterned electrochemically active region may be 0.00157 mm² to 1.57 mm², assuming a wire diameter of 20 um-400 μm and a length of 0.025 mm-2 mm. Accordingly, a surface area corresponding to a cavity region 14 that comprises a patterned electrochemically active region may be 0.00314 mm² to 3.14 mm², or 0.00471 mm² to 4.71 mm², or 0.00628 mm² to 6.28 mm², or 0.00785 mm² to 7.85 mm², or 0.00942 mm² to 9.42 mm².

The above surface area ranges correspond to a single electrochemically active area (e.g., single cavity region 14). In some embodiments, there may be a plurality of cavity regions 14 corresponding to a single sensor. In some embodiments, each of the plurality of cavity regions 14 may comprise patterned electrochemically active regions, whereas in other examples not all (e.g., one or more but less than a total number) cavity regions 14 may comprise patterned electrochemically active regions. As a representative example, assuming a 3-fold increase in surface area corresponding to a patterned vs. non-patterned cavity region 14, and a sensor that has three cavity regions 14 each of which are patterned, the sensor may have a total 3-fold increase in surface area. For a same sensor where just one of three cavity regions 14 are patterned and where two of the three cavity regions remain unpatterned, the sensor may have a total 1.67-fold increase in surface area. Such an example illustrates how particular sensors of the present disclosure may be designed with any number of cavity regions 14, where any number of such cavity regions 14 are patterned/non-patterned, to achieve a total desired electrochemically active surface area.

Accordingly, the surface patterning of the electrochemically active regions may enable an increased sensor response without a corresponding increase in sensor length and/or without a corresponding increase in the total length of the active region(s). This presents a number of advantages. As one example, sensor response may be increased by 2-3 times or greater (e.g., 4 times, 5 times, 6 times) over a sensor of equal total length and makeup (e.g., a same number and sizing of cavity regions 14) without the surface patterning of the electrochemically active surface layer. The improvement in sensor response may directly lead to an improvement in overall sensor performance. Another advantage includes a potential for reducing sensor size. For example, length of the sensors of the present disclosure may be selected depending upon depth and/or method of insertion. By employing surface patterning of electrochemically active regions, in-skin sensor length may be reduced to as little as 0.5 mm. As such, in-skin length of sensors of the present disclosure are from 0.5-10 mm. As one particular example, a sensor with an in-skin length of 10.65 mm can be reduced in size to about 2-3 mm, while still enabling a corresponding increase in sensor response by 30% or more (e.g., 35%, 40%, 50%, 60% or even higher). Other advantages include but are not limited to increasing user-friendliness in terms of alternate sensor application sites, increasing user-friendliness for pediatrics and/or pregnant women, and reducing manufacturing costs. The reduction in manufacturing cost may stem from an improved ability to achieve a consistent coating of the wire to convert it from a bare wire to a fully functioning sensor. For example, an improved (e.g., more uniform) coating can be achieved in a reduced number of steps (e.g., one step for enzyme coating as compared to multiple steps) for work pieces that have the above-described patterning of the platinum surface layer. As described herein, the term uniformity refers to the smoothness or evenness of the wire coating. A coating with greater uniformity has a smoother surface with fewer aberrations, irregularities, etc.

Moreover, it is further contemplated that dividing a single sensing region into one or more smaller cavities 14 separated by annular plates 5 (or a nub 15 and an adjacent annular plate 5) can reduce background signal arising from non-specific electrochemical activity. In such configurations, the total exposed area of the electrochemically active surface of wire 3 may be reduced even as the number of cavity regions 14 increases. This decrease in exposed surface area may diminish side reactions that contribute to background current independently of analyte presence. As a result, multi-cavity designs can be used not only to enhance analyte-specific signal but also to improve overall signal-to-noise performance and measurement stability.

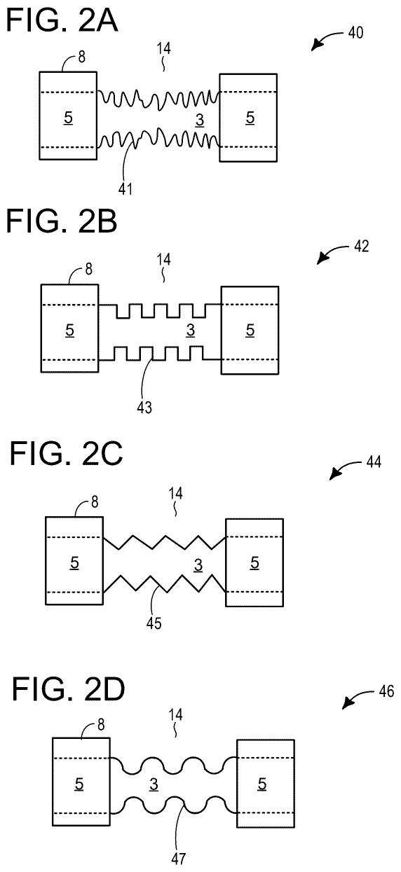

Turning to FIGS. 2A-2D, depicted are side views of portions of various work pieces shown to illustrate different types of patterning of the electrochemically active surface layer (e.g., platinum iridium surface layer) in accordance with various embodiments of the present disclosure. FIG. 2A depicts a portion of work piece 40 having a first patterning 41, which comprises an irregular approximately sinusoidal pattern. As discussed herein, irregular approximately sinusoidal patterning may be referred to as irregularly approximately sinusoidally ridged. FIG. 2B depicts a portion of work piece 42 having a second patterning 43, comprising an approximate square-wave-type (e.g., periodic square) patterning. As discussed herein, work pieces having the second patterning may be referred to as regularly squarely (or rectangularly) ridged, or irregularly squarely (or rectangularly) ridged in cases where overall height of the ridges varies substantially. FIG. 2C depicts a portion of work piece 44 having a third patterning 45, which comprises a sawtooth-type pattern. As discussed herein, work pieces having the third patterning may be referred to as regularly ridged when a height of such ridges are approximately the same, or irregularly ridged when a height of such ridges vary. FIG. 2D depicts a portion of work piece 46 having a fourth patterning, which comprises a regular sinusoidal-type patterning. As discussed herein, work pieces having the fourth patterning may be referred to as regularly ribbed when a height of the ribs is approximately the same, or irregularly ribbed when a height of individual ribs varies. Each of the above examples depicted at FIGS. 2A-2D are shown for illustrative purposes. Broadly speaking, patterning corresponding to FIGS. 2A-2C may be referred to simply as “ridged”, and patterning corresponding to FIG. 2D may be referred to simply as “ribbed.” While not explicitly illustrated, patterning of an electrochemically active surface that has any number of indents regularly or irregularly spaced may be referred to as “dimpled.” Collectively, depressions corresponding to ridges, ribs or dimples are referred to herein as “grooves.”

While FIGS. 2A-2D depict side views of portions of work pieces of the present disclosure, in other examples similar patterns may be created in the electrochemically active surface layer in circumferential fashion, similar to that discussed above with regard to FIGS. 1F-1G.

Variations of the above patterning examples are within the scope of this disclosure. Variables that may be manipulated in terms of the patterning include but are not limited to overall shape of the patterning, depth to which the patterning extends into the platinum layer, and width of each laser-ablated region corresponding to the patterning. Such variables may be accounted for when designing a sensor with particular desired sensor length, performance, manufacturing constraints, sensor robustness, etc. As examples, depth to which grooves associated with the patterning may be a percentage (or fraction) of a thickness of the surface layer (e.g., platinum iridium surface layer). For example, grooves may extend through 95%, or 90%, or 85%, or 80%, or 75%, or 70%, or 65%, or 60%, or 65%, or 60%, or 55%, or 50%, or even less, such as between 40-50%, or 30-40%, or 20-30% or 10-20%, or 1-10%, or less than 1%. Thus, as an example where the surface layer is 50 μm, a groove or grooves may be 47.5 μm deep, or 45 μm deep, or 42.5 μm deep, or 40 μm deep, and so on. A depth to which a plurality of grooves corresponding to a patterned surface layer may be variable within a cavity region 14. For example, some grooves may extend through 90% of the surface layer, whereas other grooves may extend through the surface layer to a lesser amount. Other similar examples are within the scope of this disclosure. Further, depth of grooves associated with patterning of the surface layer may vary, or be substantially the same, between cavity regions 14. For example, a particular cavity region 14 may comprise a patterning where grooves associated with the patterning extend through about 90% of the surface layer, whereas another cavity region 14 of the same sensor may comprise grooves associated with the patterning of a different depth (e.g., extending through 50% of the surface layer). Again, other similar examples are within the scope of this disclosure.

An increase in surface area resulting from patterning of the surface (e.g., platinum iridium surface) may be a function of a thickness of the surface layer. For example, an ability to increase surface area of a surface layer may be more limited as thickness of the surface layer is lessened, and may be less limited as the thickness of the surface layer is increased. Thus, starting thickness of a surface layer may be a function of a desired extent of surface area increase resulting from patterning of the surface layer. For example, surface patterning of a 50 μm thick surface layer may enable a greater surface area increase than surface patterning of a 10 μm thick surface layer, given a same starting (unpatterned) surface area. Such variables may be considered when designing sensors of the present disclosure.

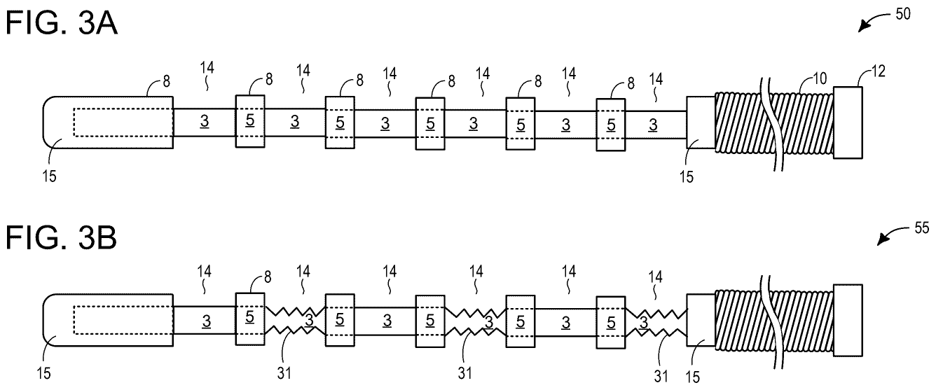

Turning now to FIGS. 3A-3B, depicted is an example of a work piece 50 (FIG. 3A), and a work piece 55 (FIG. 3B). Work piece 55 depicted at FIG. 3B may be formed from work piece 50 depicted at FIG. 3A. Specifically, FIG. 3A depicts an unpatterned work piece 50 for which insulating layer 8 has been selectively removed to yield a number of cavity regions 14. FIG. 3B depicts a work piece 55 for which the surface layer (e.g., platinum iridium layer) has been patterned. For illustrative purposes, FIG. 3B depicts an example where not all cavity regions 14 correspond to patterned surfaces. For discussion purposes, it may be appreciated that each of work piece 50 (FIG. 3A) and work piece 55 (FIG. 3B) are of a greater overall length than work piece 30 depicted at FIG. 1A.

Specifically, as discussed above one way to increase electrochemically active surface area may be to increase sensor length. There may be circumstances where an increased length is acceptable, for example for sensors designed to be placed in particular areas of the body where an increased length does not pose undesirable issues with placement. Thus, work piece 50 depicted at FIG. 3A, which as discussed is of a greater length than work piece 30 depicted at FIG. 1A, may have an increased electrochemically active surface area based solely on an increased length, in lieu of patterning, provided other variables are constant (e.g., same cavity region 14 length, similar dimensions of annular plates 5 and terminal nubs 15). For comparative purposes, assuming cavity regions 14 are of the same length between work piece 50 at FIG. 3A and work piece 30 at FIG. 1A, work piece 50 has double the number of cavities (e.g., 6 as compared to 3). If surface patterning 31 of work piece 30 at FIG. 1A results in a doubling of total electrochemically active surface area, the electrochemically active area for the shorter work piece 30 at FIG. 1A may be essentially the same as that of longer unpatterned work piece 50 depicted at FIG. 3A.

Inclusion of patterning in the surface layer (e.g., platinum iridium surface) of longer sensors may further increase electrochemically active area, and in turn, may improve sensor performance even further. FIG. 3B shows an example work piece 55, which may comprise a same overall length as work piece 50 (FIG. 3A). The work piece 55 at FIG. 3B differs from that shown at FIG. 3A by the inclusion of three different regions where the platinum surface includes patterning 31. As shown at FIG. 3B, the three regions where the electrochemically active surface layer has undergone the laser-ablation operation to produce the patterning 31 are interspersed with additional regions where the platinum surface layer has not been patterned. Thus, a sensor produced from work piece 55 may have an improved sensor performance as compared to a sensor produced from work piece 50, due to the increased active area provided via the electrochemically active surface layer patterning. FIGS. 3A-3B, taken together, illustrate how sensor performance can be fine-tuned for various sensor lengths, by selective patterning of the surface layer (e.g., platinum iridium surface layer). FIG. 3B depicts an example where the patterning is interspersed by regions of no surface patterning. However, the active area of work piece 55 may be increased even further by including the platinum layer patterning in a greater number (e.g., all) of cavity regions 14. By similar logic, the active area of work piece 55 may be decreased by including a lesser amount of surface layer patterning as compared to the illustration of FIG. 3B.

In some embodiments, the number of cavity regions 14 that include surface layer patterning may be at least partly a function of one or more of desired stability, flexibility, robustness, etc. For example, because the patterning operation results in some extent of thinning of the wire 3, there may be some compromise in terms of stability and robustness, while alternatively flexibility may at least somewhat increase as a function of a number of included patterned regions. Such variables may be important in terms of sensor insertion methodologies, resistance to sensor degradation once inserted into skin, etc. As an example, it may be desirable for a longer sensor to have regions of patterning interspersed with regions of no patterning, for increased stability/reduced flexibility. As another example, shorter sensors may not have any cavity regions 14 where there is an absence of patterning of the electrochemically active surface layer. Such examples are meant to be illustrative in nature, and are not meant to be limiting. Specifically, such examples are meant to illustrate that patterning of one or more cavity regions of a work piece as herein disclosed, may impart an ability to precisely manipulate sensor performance as a function of overall sensor length, depending on the application. In particular, sensors can be manufactured with one or more shorter individual cavities, such that annular plates 5 or nubs 15 are positioned more closely together to achieve comparable or superior analyte sensing profiles compared to longer sensors with fewer, larger cavities. This approach can enable reduction of in-skin sensor length without sacrificing performance, which may offer design benefits for comfort, anatomical compatibility, and user experience.

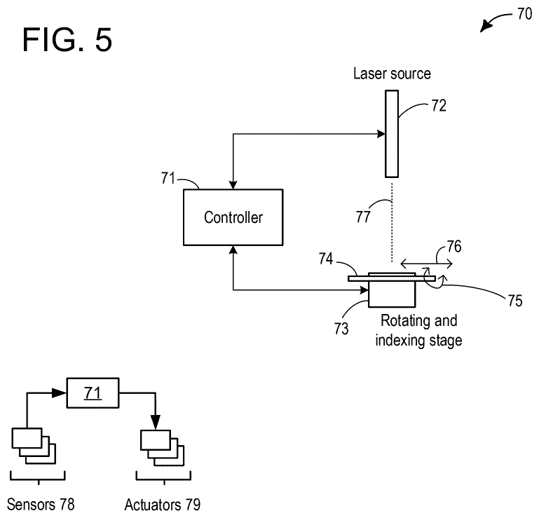

Turning now to FIG. 5, depicted is a high-level example system 70, for use in producing work pieces of the present disclosure. The work pieces in turn may be used to create the analyte sensors of the present disclosure.

Example system 70 at FIG. 5 includes a controller 71. Controller 71 may be capable of receiving one or more signals from one or more sensors 78, and may additionally or alternatively be capable of sending one or more signals to one or more actuators 79, to at least partially create the electrochemically active surface layer patterning (e.g., platinum iridium surface layer patterning) of work pieces of the present disclosure.

Example system 70 includes laser source 72, and rotating and indexing stage 73. Rotating and indexing stage 73 may be configured to circumferentially rotate (refer to arrow 75) a work piece 74 of the present disclosure and additionally move the work piece 74 length-wise (refer to arrow 76). Laser source 72 may be capable of femtosecond laser pulses 77, although faster or slower pulses are within the scope of this disclosure. Thus, example system 70 may include any number of sensors and any number of actuators for carrying out the laser-based patterning of the work pieces of the present disclosure. Controller 71 may receive input from the various sensors, process the input data, and trigger the actuators in response to the processed input data based on instructions or code programmed therein corresponding to one or more routines (e.g., a routine that encompasses all or part of methodology discussed herein).

Controller 71 may be communicably coupled to sensors and/or actuators of system 70 via one or more of a wireless network, wired network, or a combination of wired and wireless networks (exemplified via unlabeled arrows at FIG. 5). Suitable networks include, but are not limited to, the Internet, a personal area network, a local area network (LAN), a wide area network (WAN) or a wireless local area network (WLAN), for example. Network devices (not shown) may include local area network devices such as routers, hubs, switches, or other computer networking devices. Instructions for controlling the one or more routines may be, for example, user-defined. In this way, a user may exert control over a desired surface layer patterning, extent of patterning, overall shape of patterning, pattern depth, etc., depending on the application for the particular work piece and ultimately, assembled sensor.

In some embodiments, example system 70 may be used for both laser ablating the insulating layer (e.g., insulating layer 8) as well as laser-ablating the electrochemically active surface layer (e.g., platinum iridium surface layer), however in other examples different systems may be used for each separate task. For example, one of chemical etching, grit-blasting, excimer lasing, manual removal, etc., may be used for removal of the insulating layer 8, whereas example system 70 may be used for laser-ablation of the electrochemically active layer.

In some embodiments where laser-ablation is relied upon to ablate the insulating layer as well as to create the electrochemically active surface layer patterning, a laser source (e.g., laser source 72 at FIG. 5) may be either the same or may be different. Furthermore, a different laser wavelength and/or pulse duration may be relied upon for ablating the insulating layer as compared to creating the patterning in the electrochemically active surface layer. However, in other examples, a same laser wavelength and/or pulse duration may be used for both ablating the insulating layer as well as creating the patterning in the electrochemically active surface layer. In some embodiments, the removal of the insulating layer and the patterning can be combined into one operation. The same laser can be used to ablate the insulation and the platinum. The ablation process is typically done in several steps. The ablation done to the insulation may be limited/constrained by reducing the power of the laser. In some embodiments, the ablation is performed using an ultraviolet (UV) laser.

Example system 70 is meant to be illustrative, and other similar systems for production of work pieces with the desired platinum layer patterning are within the scope of this disclosure.

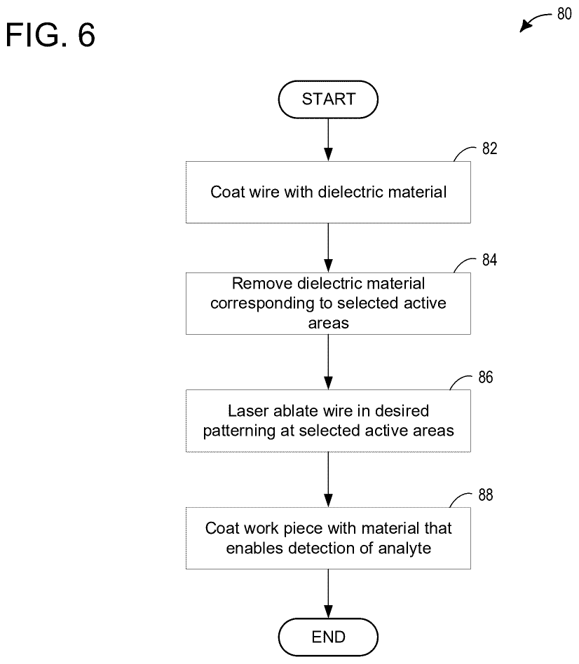

Turning now to FIG. 6, a high level example method 80 is shown, for producing biosensors of the present disclosure. In some embodiments, at least a portion of method 700 may be conducted under control of a controller, for example controller 71 of example system 70 depicted at FIG. 5.

Method 80 begins at 82, and includes coating the wire (e.g., tantalum core wire coated with platinum iridium layer) with a dielectric material (e.g., polyimide layer). Proceeding to 84, method 80 includes removing select regions of the dielectric material, to expose the electrochemically active surface layer (e.g., platinum iridium layer). As discussed above, removal of select regions of the dielectric material may comprise one or more of laser-ablation, chemical etching, grit-blasting, excimer lasing, manual removal, etc. In some embodiments, the wire 3 is passed through a Hylax Laser Skiving system to create an ablation pattern that can provide analyte (e.g., glucose) sensing cavities and/or solder points in sensor 35 manufacturing. Each removed region (e.g., cavity region 14) may be 0.025-3.5 mm in length, depending on the application. For example, in an application including an insertion angle of about 90°, the sensing electrode can be relatively short (e.g., about 2 mm) and include multiple (e.g., four) cavities of a shorter length (e.g., about 0.125 mm) and annular plates (e.g., annular plates 5) dispersed therebetween. Such configurations can provide sufficient or superior sensitivity per mm when the surface area of the cavity is decreased. Removal of the dielectric material in this fashion may result in any number of annular plates (e.g., annular plates 5 at FIGS. 1A-1B) being created on the work piece for which method 80 is being used to produce, and may impart the work piece with two terminal nubs (e.g., terminal nubs 15).

With the dielectric material having been removed in the select regions, at 86 method 80 includes laser ablating the electrochemically active surface layer at one or more of the regions (e.g., cavity regions 14 at FIGS. 1A-3B) for which the electrochemically active surface layer has been exposed. As mentioned, in some embodiments, system 70 depicted at FIG. 5 may be utilized at 86 for creating a desired patterning in the electrochemically active surface layer. Next, at 88, method 80 includes coating the work piece with material (e.g., membrane material 22 at FIG. 1B) that enables detection of a particular analyte, to produce a biosensor of the present disclosure. In the case of a glucose sensor (e.g., continuous glucose monitor), the coating at 88 of method 80 may include at least a glucose oxidase layer.

The methodology discussed above includes steps of coating a non-patterned wire with dielectric material, selectively removing particular regions of the dielectric material to yield any number of cavity regions that each expose an electrochemically active surface layer, and then conducting a surface layer patterning operation to create a desired patterning in one or more of the cavity regions. In some embodiments, the electrochemically active surface layer (e.g., platinum iridium surface layer) may be patterned prior to the wire being coated with the dielectric material.

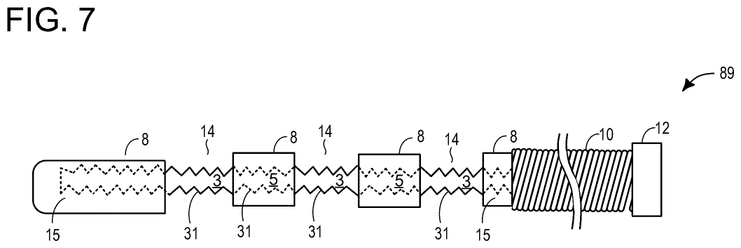

Turning to FIG. 7, depicted is yet another work piece 89 for which the surface layer has been patterned (e.g., via the system of FIG. 5) prior to wire 3 being coated with dielectric material 8. Subsequent to the coating of wire 3 with dielectric material 8, removal of select portions of dielectric material 8 creates cavity regions 14, thereby exposing electrochemically active surface area of wire 3 that already includes patterning 31. As depicted, patterning 31 extends through each of annular plates 5 and terminal nubs 15.

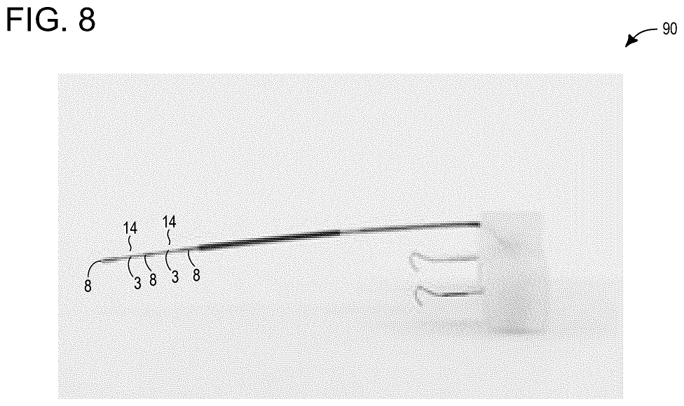

Turning to FIG. 8, depicted is a digital image of a work piece of the present disclosure, shown to illustrate a work piece subsequent to laser ablating the dielectric material but prior to creating the platinum layer patterning as discussed herein. Labeled at FIG. 8 are thus cavity regions 14, and insulating layer 8. Exposed wire 3 corresponds to cavity regions 14 where insulating layer 8 has been removed.

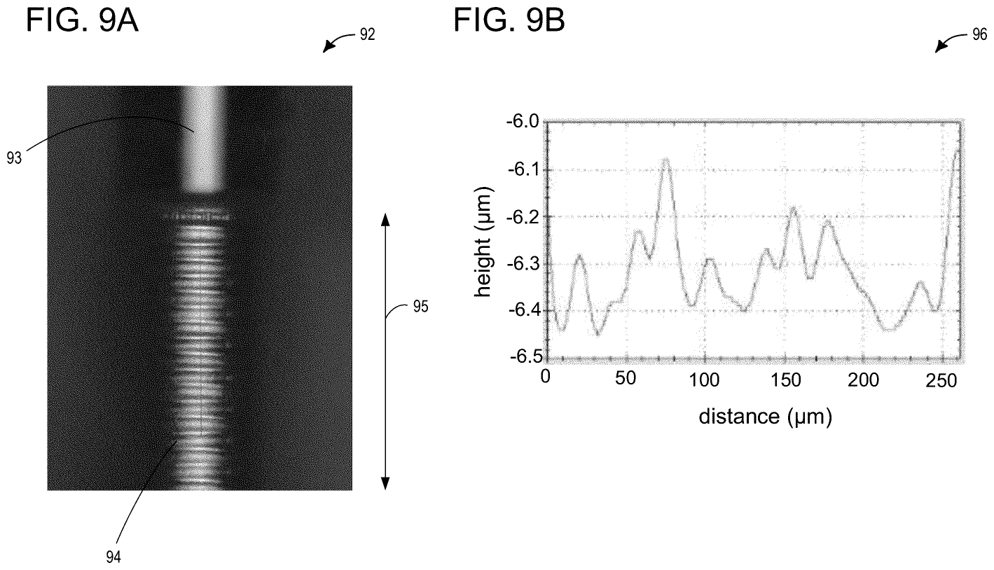

Turning now to FIGS. 9A-9B, depicted is an example of a portion of an actual work piece 92 of the present disclosure (FIG. 9A) illustrating platinum iridium layer patterning, along with a graph 96 (FIG. 9B) depicting dimensional measurement of the patterning of the portion of the work piece depicted at FIG. 9A. Specifically, at FIG. 9A, a first portion 93 of the work piece 92 corresponds to a region where the dielectric material has not been ablated. A second portion 94 of the work piece 92 depicts a cavity region (e.g., cavity region 14 at FIG. 2A) following laser ablation of the platinum iridium layer to yield a desired patterning of the platinum iridium layer. Graph 96 at FIG. 9B depicts distance (in μm) along a length of a portion of work piece 92. For reference, distance at graph 96 corresponds to the length of work piece 92 as defined by line 95 at FIG. 9A. In this particular example, the distance shown corresponds to 0.25 mm. However, as discussed above, cavity regions 14 may be significantly smaller than 0.25 mm, for example 0.1 mm or less (e.g., 0.05 mm, or 0.025 mm). Example system 5 may be adjusted in such examples to create a similar patterning as that illustrated at FIG. 9B. For example, indexing steps may be adjusted so as to enable patterning of small cavity regions (e.g., cavity regions as small as 0.025 mm), in similar fashion as that depicted at FIGS. 9A-9B. Height (in μm) refers to depth to which the platinum iridium layer has been ablated along length 95 of work piece 92. In the example depicted at FIG. 9B, maximum depth of patterning approximates about 300 nm, however it is within the scope of this disclosure to create deeper grooves within the electrochemically active surface layer. In some embodiments, the grooves can go all the way down to the tantalum core wire. Examples have been provided above. By creating the patterning in the platinum iridium layer via the methodology herein disclosed, area of the work piece 92 corresponding to the cavity region is increased as compared to a similar work piece for which the platinum iridium layer does not include any patterning.

Specifically, the patterning of the platinum iridium layer as herein disclosed may enable an increase in sensor response by 2-3 times over a similarly constructed sensor in an absence of the patterning. As discussed herein, sensor response improvements may correspond to one or more of a reduction in signal-to-noise, increase in measured current associated with analyte detection, and decrease in timeframe to accurately acquire analyte signals. In terms of sensor signal, the patterning techniques described herein allow for more frequent and accurate results, as well as shorter signal smoothing and lower lag time. The patterning techniques may thus result in more consistent sensors and more accurate factory calibration systems that are stable for longer periods of time. This can directly lead to an improvement in the performance of the sensor system (e.g., CGM device). As one example, for each 0.2 nAmp/mM of in vivo analyte (e.g., glucose), performance of sensors as measured by a parametric such as mean absolute relative difference (MARD) and/or mean absolute difference (MAD) can be improved (e.g., decreased) by 1% or more.

Because, as disclosed herein, it is possible to increase sensor response without increasing sensor length (and even increase sensor response while correspondingly reducing sensor length), it is herein recognized that it may be possible to reduce sensor size without compromising performance. As one particular example, in-skin length can be reduced to 5 mm or less, while still increasing sensor response by 30% or more. This in turn may enable application of the sensor to many more locations on the body, in particular the arm. The reduction in in-skin length may additionally be advantageous in terms of pediatric use and use with pregnant women, among others. Other advantages include but are not limited to improving coating of the work piece with the material that enables analyte detection, which in turn may reduce manufacturing time and costs associated with manufacturing.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of constructing an indwelling analyte sensor, the method comprising:
applying a dielectric material to a wire comprising electrochemically active surfaces;
removing one or more selected regions of the applied dielectric material to expose one or more electrochemically active surfaces of the electrochemically active surfaces of the wire;
producing a pattern in at least one of the exposed one or more electrochemically active surfaces; and
applying a membrane material to remaining regions of the dielectric material and the exposed one or more electrochemically active surfaces, the membrane material comprising an enzyme layer.

2. The method of claim 1, wherein:
the indwelling analyte sensor comprises a glucose sensor; and
the enzyme layer comprises a glucose oxidase layer.

3. The method of claim 1, wherein the dielectric material comprises at least one of a polyimide material, a biocompatible solder mask material, an epoxy acrylate copolymer material, a polyurethane material, or a parylene material.

4. The method of claim 1, wherein removing the one or more selected regions of the dielectric material comprises at least one of manual removal, laser-ablation, chemical etching, or grit-blasting the one or more selected regions of the dielectric material to expose the one or more electrochemically active surfaces of the wire.

5. The method of claim 1, wherein the wire comprises a tantalum core wire coated with a platinum iridium layer.

6. The method of claim 5, wherein producing the pattern comprises performing a laser-ablation operation of the platinum iridium layer corresponding to at least one of the one or more electrochemically active surfaces.

7. The method of claim 6, wherein performing the laser-ablation operation comprises laser-ablating the platinum iridium layer with a femtosecond laser source.

8. The method of claim 6, wherein performing the laser-ablation operation comprises rotating and indexing the wire.

9. The method of claim 1, wherein producing the pattern comprises creating a plurality of grooves in the electrochemically active surface.

10. The method of claim 9, wherein each of the plurality of grooves has a depth of at least 20 nm.

11. The method of claim 1, wherein applying the membrane material comprises brush coating the remaining regions of the dielectric material and the one or more electrochemically active surfaces with the enzyme layer.

12. The method of claim 1, wherein the pattern increases a sensitivity per mm of the one or more exposed electrochemically active surfaces as compared to electrochemically active surfaces lacking the pattern.

13. An indwelling analyte sensor, comprising:
an electrochemically active surface coated with a dielectric material;
at least one cavity region in which the electrochemically active surface is free of the dielectric material and wherein the electrochemically active surface is of a patterned texture in one or more of the at least one cavity regions, the patterned texture increasing a surface area of the electrochemically active surface in the at least one cavity region as compared to a non-patterned electrochemically active surface in the at least one cavity region; and
a membrane material comprising an enzyme layer, the enzyme layer surrounding the dielectric material and the electrochemically active surface corresponding to the at least one cavity region.

14. The sensor of claim 13, wherein the dielectric material is a polyimide material.

15. The sensor of claim 13, wherein the electrochemically active surface comprises a platinum iridium layer.

16. The sensor of claim 15, wherein the platinum iridium layer surrounds a tantalum core wire.

17. The sensor of claim 13, wherein the membrane material includes multiple membranes.

18. The sensor of claim 13, wherein an in-skin length of the sensor is 7 mm or less.

19. The sensor of claim 13, wherein the patterned texture comprises one or more of a ribbed texture, a ridged texture, and a dimpled texture.

20. A glucose biosensor, comprising:
a set of two terminal nubs of dielectric material extending outward from and spaced along an electrochemically active surface;
one or more cavity regions defined by an absence of dielectric material, the one or more cavity regions positioned between the set of two terminal nubs;
a membrane material comprising a glucose oxidase layer that surrounds the set of two terminal nubs and the electrochemically active surface; and
wherein the electrochemically active surface extends through at least a portion of the set of two terminal nubs, and has a patterned surface texture in at least the one or more cavity regions, the patterned surface texture increasing a surface area of the electrochemically active surface corresponding to the one or more cavity regions as compared to an electrochemically active surface lacking the patterned surface texture.

21. The biosensor of claim 20, wherein the electrochemically active surface comprises a platinum iridium layer surrounding a tantalum core wire; and
wherein the platinum iridium layer comprises the patterned surface texture.

22. The biosensor of claim 20, wherein the one or more cavity regions each have a length of 0.025 mm-3 mm.

23. The biosensor of claim 20, wherein the membrane material has an outer surface, the outer surface defining a concave curve curving toward the electrochemically active surface in the one or more cavity regions; and
wherein the membrane material includes multiple membranes and defines an external surface of the biosensor.

24. The biosensor of claim 20, further comprising a plurality of annular plates spaced between the set of two terminal nubs.

25. The biosensor of claim 24, wherein:
the one or more cavity regions are positioned (i) between at least two of the plurality of annular plates and (ii) between at least two of the plurality of annular plates and the set of two terminal nubs;
the glucose oxidase layer surrounds the plurality of annular plates; and
the electrochemically active surface extends through at least two of the plurality of annular plates.

26. An indwelling analyte sensor, comprising:
an electrochemically active surface devoid of a dielectric material;
at least one cavity region in which the electrochemically active surface is of a patterned texture, wherein the patterned texture increases a surface area of the electrochemically active surface in the at least one cavity region relative to a corresponding non-patterned electrochemically active surface; and
a membrane material comprising an enzyme layer that surrounds the electrochemically active surface in the at least one cavity region, wherein the patterned texture of the electrochemically active surface increases a uniformity of the enzyme layer surrounding the electrochemically active surface relative to a non-patterned texture.

27. The sensor of claim 26, wherein the uniformity of the enzyme layer corresponds to a smoothness of the enzyme layer surrounding the electrochemically active surface.
